(19) United States
(12) Patent Application Publication
(10) Pub. No.: US 2008/0242917 A1
(43) Pub. Date: Oct. 2, 2008

(54) Delivery and Deployment Device For Surgical Web

(76) Inventor: George Kaladelfos, New South Wales (AU)

Correspondence Address: STINSON MORRISON HECKER LLP;ATTN: PATENT GROUP, 1201 WALNUT STREET, SUITE 2800, KANSAS CITY, MO, 64106-2150, US

(21) Appl. No.: 11/631,441

(22) PCT Filed: Jul. 8, 2005
(86) PCT No.: PCT/AU05/01007

§ 371 Date: Dec. 10, 2007

(30) Foreign Application Priority Data
Jul. 9, 2004 (AU) 2004903793

(51) Int. Cl. A61F 2/02

(52) U.S. Cl. 600/30

(57) ABSTRACT

A device (10) for delivery and deployment of an elongate web into a patient, the device comprising a generally elongate carrier member (20) adapted to be inserted into a body cavity or tissue of a patient, the carrier member having a proximal end (23) and a distal end (21), and a passage (28) extending along the carrier member at least part way along its length; and an elongate deployment member (30) having a distal end (32) and a proximal end (34) and being shaped and configured to be located in the passage and to be moved axially along the passage from a position adjacent the proximal end of the carrier member. A web is able to be housed in said passage when said deployment member is located in the passage and deployed from the passage into a patient with axial movement of the deployment member relative to the carrier member.

### FIELD OF THE INVENTION

The present invention relates to a device for delivery and deployment of a web within the body of a patient, more particularly, but not exclusively in the field of obstetrics and gynecology and more particularly the invention relates to an apparatus and method for the treatment of female urinary incontinence.

### BACKGROUND OF THE INVENTION

Female incontinence often occurs due to deterioration of or at least partial damage to ligaments and muscles tissue in the pelvic region. Often involuntary leakage of urine results due to lack of patient control of the pelvic muscles, particularly the urogenital diaphragm and pubo-urethral ligaments. It has been found that the condition may be treated by creating some form of artificial ligament between the pelvic muscles and the vagina, with the ligament passing behind the pubis.

Lack of anterior support may be corrected by techniques such as use of a suburethral sling, more specifically by use of tension-free-tape whereby the sling is not sutured is a techniques commonly used. This technique requires two 5 mm incisions suprapubically and a 2 cm vaginal incision under the mid-portion of the urethra, and the threading of a tape material under the urethra.

The application is aware of two prior art patents which deal with similar subject matter to that of this invention, namely U.S. Pat. No. 5,112,344 Petros and U.S. Pat. No. 5,899,909 Claren, both of which require upon a loop or sling type arrangement for correction and/or support of anatomical structures.

U.S. Pat. No. 5,112,344 describes a method and apparatus for treating female incontinence. The surgical instrument for the application of a filamentary element into the body comprises a tubular shaft having a handle at one end and a flexible needle slidably receivable in the shaft and adapted at one end to receive a filamentary element. The method of treating female incontinence comprises looping a filamentary element between the wall of the vaginal and the rectus abdominis sheath in the anterior wall of the abdomen whereby it masses to each side of the urethra, adjusting the loop to bring the vaginal wall and the urethra into the correct spatial relationship to the pubis allowing the development of scar tissue between the vaginal wall and the anterior wall of the abdomen pubic symphysis and removing the filamentary element.

The Claren Patent, U.S. Pat. No. 5,899,909, also describes a surgical instrument and a method for treating female urinary incontinence. The instrument comprises a shank having a handle at one end thereof, and two curved needle-like elements which are connected at one end thereof each with one end of a tape intended to be implanted into the body. These elements can be connected one at a time with the shank at the other end thereof to form a curved end portion of the shank and are intended to be passed into the body via the vagina, each element being dimensioned to extend from the inside of the vaginal wall over the back of the pubic bone to the outside of the abdominal wall. When practicing the method the tape is passed into the body via the vagina first at one end and then at the other end at one side and the other, respectively, of the urethra to form a loop around the urethra, located between the urethra and the vaginal wall. The tape is extended over the pubis and through the abdominal wall and is tightened. Then, the tape ends are cut at the abdominal wall, and the tape is left implanted in the body.

The present inventor, having regard to the teachings and shortcomings of the prior art, for example the techniques and devices as discussed above, has surprisingly found that stress induced incontinence need not be treated by a loop or sling-type structure or slinging method and as such, deficiencies and shortcomings of the prior art have been substantially ameliorated by the present invention as follows.

Any discussion of documents, publications, acts, devices, substances, articles, materials or the like which is included in the present specification has been done so for the sole purpose so as to provide a contextual basis for the present invention. Any such discussions are not to be understood as admission of subject matter which forms the prior art base, or any part of the common general knowledge of the relevant technical field in relation to the technical field of the present invention to which it extended at the priority date or dates of the present invention.

### SUMMARY OF THE INVENTION

In broad terms, the present invention is a device for delivery and deployment of an elongate web into a patient, the device comprising:

a generally elongate carrier member adapted to be inserted into a body cavity or tissue of a patient, the carrier member having a proximal end and a distal end, and a passage extending along the carrier member at least part way along its length; and

an elongate deployment member having a distal end and a proximal end and being shaped and configured to be located in the passage and to be moved axially along the passage from a position adjacent the proximal end of the carrier member;

wherein the relative shape and configuration of the passage and the deployment member are such that a web is able to be housed in said passage when said deployment member is located in the passage; and

axially moving the carrier member and deployment member relative to each other in use being adapted to cause the web to be deployed from the passage into a patient.

In an embodiment of the invention, the carrier member is preferably of generally tubular configuration. Preferably an engagement means is formed on or adjacent the distal end of said deployment member for engaging with the web and enabling the deployment member to carry the web out of the passage as the carrier member and deployment member are moved relative to each other. The engagement means of said deployment member is preferably adapted to disengage from the web when the deployment member is retracted from the patient in use.

The carrier member may be circular in cross-sectional shape. Alternatively, the carrier member may be ovoid or elliptical in cross-sectional shape.

The carrier member may be curved at least part way along its length, and the deployment member may be laterally flexible.

Preferably, the carrier member and said deployment member are adapted to be locked together to prevent relative movement during insertion of the carrier member into a patient.

In another embodiment, the device may further comprise a generally elongate breach member, said breach member adapted to slidingly engage with said carrier member and extend away from the proximal end of the carrier member. Preferably the deployment member and said breach member are adapted to be locked together.

The passage may be an open channel and said deployment member has a portion inclined to the longitudinal axis of the carrier member such that relative motion between said carrier member and said deployment member drives the web member from the passage of the carrier member.

The elongate carrier member may have a cutting tip formed at the distal end thereof.

In another embodiment, the device further comprises an elongate cutting member axially slidable in the passage of said carrier member so as to extend beyond the distal end of said carrier member, said cutting member having a distal end having cutting tip formed thereon, and a proximal end, the cutting member being removable from said passage after the carrier member has been operatively inserted into a patient in use.

The cutting tip of said cutting member preferably includes at least one pair of opposite faces extending away from the proximal end of the cutting member and meeting at an apex.

Preferably, the device is adapted to deploy a web elongate in shape and formed from a biocompatible polymeric material. The web is preferably textured so as to allow ingrowth of tissue.

The present invention extends to method for treating incontinence in a patient, the method comprising the steps of:

positioning a first elongate web adjacent to the pubo-urethral ligament of a patient on a first side; and

positioning a second elongate web adjacent to the pubo-urethral ligament of a patient on the contra-lateral side to the first elongate web;

wherein the first and the second elongate webs are formed by a material having a texture so as to allow ingrowth of tissue; and

the first and the second elongate webs are positioned at paraurethral locations within the body of the patient.

Preferably the first web and the second web are introduced into the body of the patient through a single access incision. The first web and the second web are preferably introduced into the body with a device according to the present invention.

The present invention further extends to a method of treating a patient by positioning an elongate web of material at a selected location within the patient, the method including the steps of:

inserting a generally tubular carrier member into the patient, the carrier member having a proximal end and a distal end and a deployment passage extending between said ends, the carrier member being inserted to a position in the patient such that the distal end is at or adjacent said selected location; and

deploying said elongate web to said selected location through said deployment passage, using an elongate deployment member passing down said passage.

Preferably the step of deploying said elongate web is carried out by positioning said web at the distal end of the carrier member, keeping the web at that position by holding the deployment member stationary, at least partially withdrawing the carrier member from the patient to thereby leave the deployment member and web at said selected location, and fully withdrawing the carrier member and deployment member from the patient, leaving the web in said selected location within the patient.

The selected location may be the muscle of the vaginal wall of a patient, and the method is preferably for treatment of patient having vault or vaginal prolapse.

Alternatively, the selected location is the pubu-urethral ligament of a patient, and the method is preferably for treatment of a patient having stress-induced incontinence.

The elongate web is preferably delivered to and deployed at said selected location using a device according to the present invention. Preferably the elongate web is adapted to allow tissue ingrowth. The elongate web is preferably provided with mechanical properties so as to augment the tissue of the patient at the selected location. The elongate web may include at least one pharmaceutical substance for delivery of said pharmaceutical to said selected location.

The present inventor has found that incontinence in a female patient is treatable by deploying an individual elongate web-like structure para-urethrally and bilaterally in a patient. The device of the present invention provides for suitable deployment of a web for treatment of urinary incontinence. The present invention is distinguished from devices and methods of the prior art as it does not require the use of a sub-urethral sling, or a method or device for implanting such a sling. As such, according to the present invention, a single entry point may be used for the deployment of appropriate web members for treatment of such a complaint. Furthermore, unsightly scarring by suprapubic incisions is absent in the technique of the present invention. Ease of deployment, reduced operating time and easier post-operative management addresses at least some of the disadvantages exhibited in the prior art techniques, whilst addressing a complaint presented by many female patients.

These and other advantages of the present invention will become apparent reading the following description of preferred embodiments thereof given below by way of example only. In the detailed description, reference is made to the accompanying drawings, however specific features depicted within the drawings should not be construed as limiting the invention.

Throughout the specification the term “comprise” and variations on this term including “comprising” and “comprises” are to be understood to imply the inclusion of a feature, integer, step or element, and not exclude other features, integers, steps or elements.

### BRIEF DESCRIPTION OF THE DRAWINGS

The invention will now be described, by way of example only, and with reference to the accompanying drawings in which:

FIG. 1 depicts a perspective view of an embodiment of a device according to the invention;

FIG. 2a-2d depict progressive deployment of a web from the device of FIG. 1;

FIG. 3a shows a distal end view of a first embodiment of the present invention;

FIG. 3b shows a distal end view of a second embodiment of the present invention;

FIG. 3c shows a perspective view of the embodiment depicted by FIG. 3b;

FIG. 3d shows a longitudinal sectional view of the embodiment depicted in FIG. 3c;

FIG. 3e shows a distal end view of the embodiment depicted in FIGS. 3c and 3d;

FIG. 4a shows a first embodiment of engagement of the present invention with a surgical web;

FIG. 4b shows a second embodiment of engagement of the present invention with a surgical web;

FIG. 4a shows a third embodiment of engagement of the present invention with a surgical web;

FIG. 5 depicts an embodiment of the device according to the invention;

FIG. 6 depicts a further embodiment of the device according to the invention;

FIG. 7a shows a perspective view of a still further embodiment of the device according to the invention;

FIG. 7b shows a cross-sectional view of the embodiment depicted in FIG. 7a;

FIGS. 8a and 8b show an example of a use for the present invention; and

FIG. 9 shows a lateral view of use of the present invention.

### DETAILED DESCRIPTION OF THE EMBODIMENTS

The following description refers to preferred embodiments of a device for delivery and deployment of an elongate web into a patient according to the present invention. To facilitate an understanding of the invention, reference is made in the description to the accompanying drawings whereby the device is illustrated in preferred embodiments. Similar components between the embodiments are identified by the same reference numerals. It will be understood that although described with reference to particular surgical procedures, the device may be used for other or alternate surgical procedures without departing from the spirit and scope of the invention.

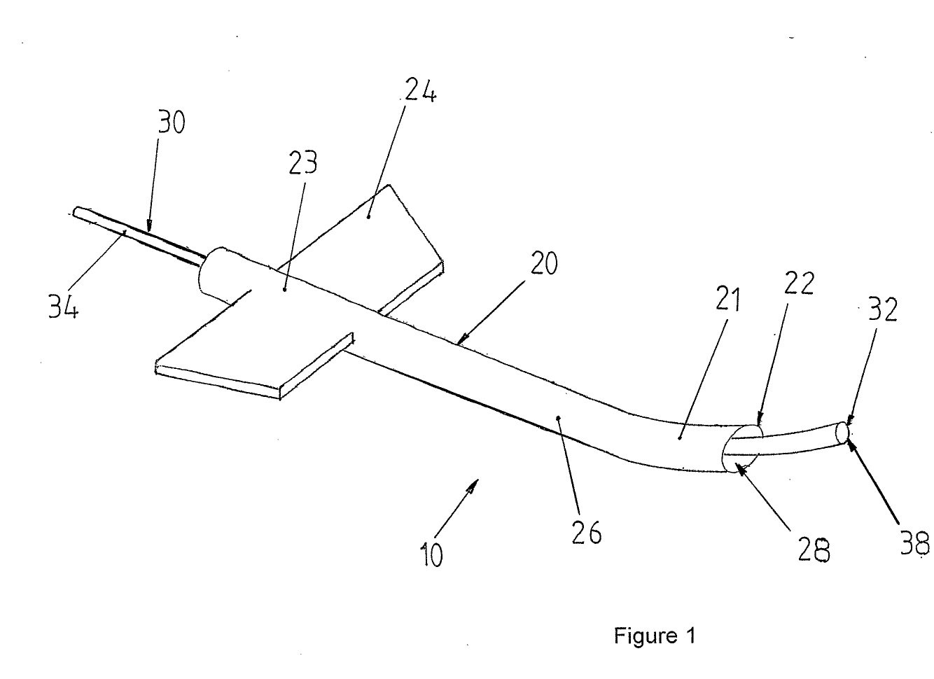

Referring to FIG. 1, there is shown an embodiment of the device 10 according to the present invention having a generally elongate carrier member 20 which has a cutting tip 22 located at a distal end 21, and a handle means 24 located at the proximal end 23 thereof. An elongate deployment member 30 is located within the passage 28 of the carrier member 20. In the present embodiment, the deployment member 30 has a cutting tip 38 located at the distal end 32 thereof. The deployment member 30 can move axially within the passage 28. The distal end 21 of the carrier member 20 may optionally be curved as shown depending upon the application for which the device is to be used. Optionally, depending upon the degree of curvature, the deployment member 30 may also be curved or may be flexible such that it may pass through the passage 28.

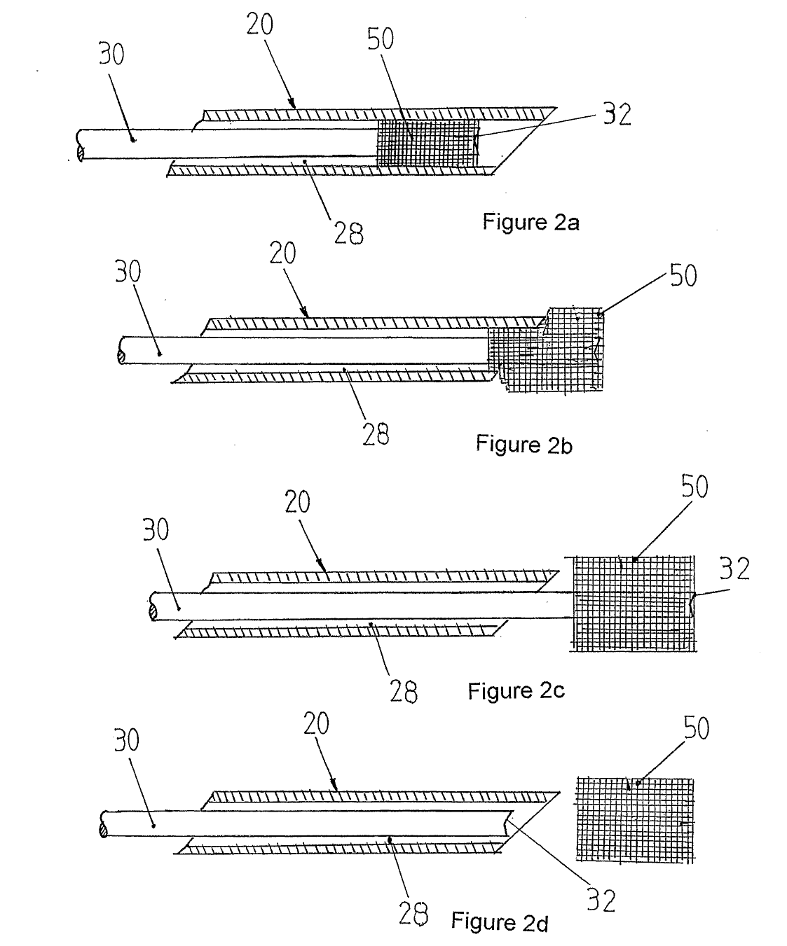

FIG. 2 shows a cross sectional view of an embodiment of the present invention at the distal end of the device 10 whereby a web 50 is progressively deployed from a carrier member 20. In FIG. 2a the web 50 is shown to be folded around the deployment member 30, wholly housed with the passage 28 and is detachably engaged with the distal end 32 of the deployment member 30. As the deployment member 30 is advanced distally with respect to the carrier member 20 as shown in FIG. 2b, the web 50 is deployed from the carrier member 20 and at least partially unfolds. Further distal advancement as shown in FIG. 2c causes the web 50 to be further deployed from the carrier member 20 and unfold until the web 50 is fully deployed from the carrier member 20 and in an unfolded state. Proximal advancement of the deployment member 30 relative to the carrier member 20 causes the web 50 to be disengaged from the distal end 32 of the deployment member 30 as shown in FIG. 2e.

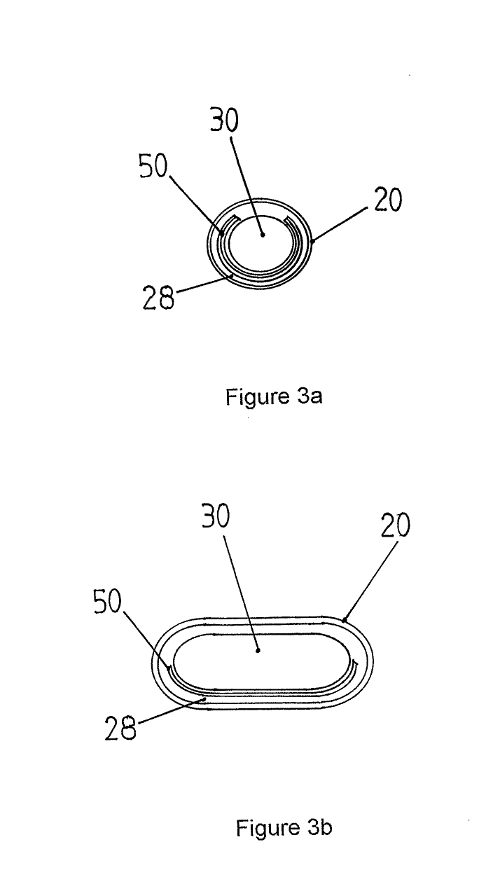

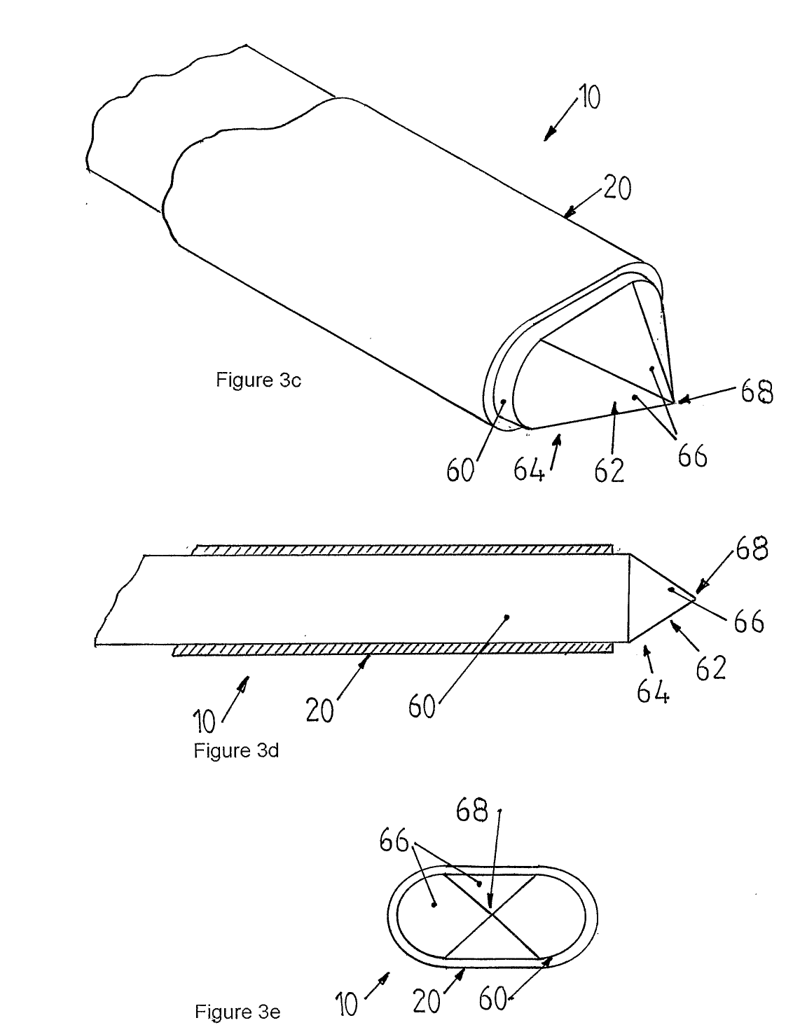

FIGS. 3a and 3b depicts examples of the cross-sectional shape of the carrier member 20 and the deployment member 30 toward the distal end of the device according to FIG. 1. A web 50 is shown to be disposed between the carrier member 20 and the deployment member 30. The clearance provided between the carrier member 20 and the deployment member 30 is such that the web 50 will not be damaged or cause jamming whilst being advanced through the passage 28.

FIG. 3a shows a circular cross-section whereby the web 50 is arranged around the deployment member 30 in a circular fashion. FIG. 3b shows a further example of a suitable cross-sectional shape of the carrier member 20 whereby the cross-section shape is ovoid or elliptical and the deployment member 30 which may be used for delivery of the web 50.

Referring to FIGS. 3c, 3d and 3e there is shown an embodiment of the invention wherein the carrier member 20 has an ovoid cross-sectional shape and the distal end 21 of the carrier member 20 is perpendicular to the longitudinal axis thereof.

In the present embodiment, the device 10 further comprises a cutting member 60 also of ovoid cross-sectional shape which is sized so as to be located within the passage 28 of the carrier member 20. The cutter member is slidable within the passage 28 and includes a cutting tip 62 located at a distal end 64 thereof. In this embodiment, the cutting tip 62 of the cutting member 60 is defined by two pairs of opposite faces 66 which extend to an apex 68. It will be appreciated that the cutting tip 62 may be configured in alternate embodiments without departing from the scope of the invention.

Prior to insertion of the carrier member 20 within the body of a patient, the cutter member 60 is positioned within the passage of the carrier member 20 such that the cutting tip 62 of the cutting member 60 protrudes beyond the distal end 21′ of the carrier member 20. A locking means, although not depicted within the present embodiment, may be provided such that relative motion between the carrier member 20 and the cutting member 60 is prevented.

During insertion of the carrier member 20 within the body of a patient, the cutting tip 62 of the cutting member 60 parts tissue of the patient so as to allow the distal end 21 of the carrier member 20 to be advanced to a predetermined surgical site. The faces 66 of the cutting tip 62, as arranged in the present embodiment, allow the carrier member to be readily advanced through the tissue without the cutting member 60 and hence the carrier member 20 veering in an undesired direction. This provides ease of use of the device 10 by a surgeon when advancing the distal end 21 of the carrier 20 to the surgical site.

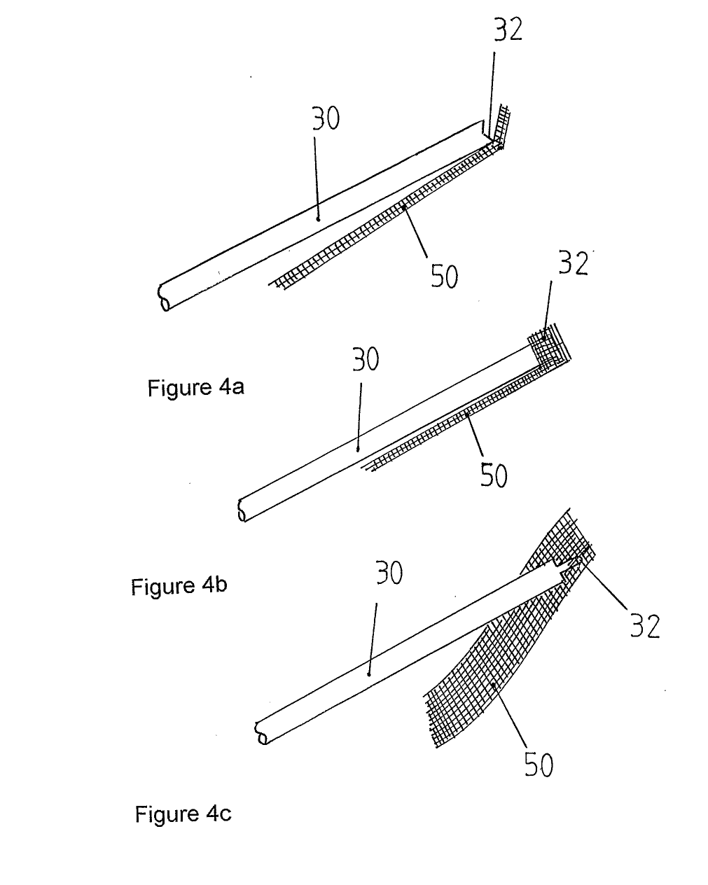

Upon location of the distal end 21 of the carrier member 20 at the predetermined surgical site, the cutting member 60 may be retracted by the surgeon through the passage 28 of the carrier member 20. Upon retraction of the cutting member and removal of the cutting member 60 from the passage 28 of the carrier member 20, a deployment member having a surgical web 30 attached to the distal end thereof as described below with reference to FIGS. 4a-4c to be advanced through the passage 28 of the carrier member 20, and deployed from the distal end 21 at the surgical site in a manner as described with reference to FIGS. 4a-4c.

FIG. 4 depicts various examples of manners in which a web 50 may be engaged with a deployment member 30. FIG. 4a shows a side view of a web 50 engaged with a deployment member 30. In this example, the deployment member 30 has a protruding portion at the distal end 32 which engages with the texture of the web 54. Another example is depicted in FIG. 4b whereby as web 50 is folded over the distal end 32 of a deployment member 30 so as to form a sock-type arrangement. The web 50 may be tacked back upon itself by an adhesive to form the sock-type arrangement or alternatively sutured or hot melted. It will be appreciated that numerous alternate manners exist in which the function of the sock-type construct is achieved. In FIG. 4c, the web 50 has a small aperture formed in it so as to allow a protruding portion of the distal end 32 of the deployment member 30 to extend into the aperture so as to secure the web to the deployment member. It must also be appreciated that the manners in which the wed 50 is attached to the deployment member 30 in each of FIGS. 4a, 4b and 4c allows for the web 50 to be readily disengaged from the deployment member 30 upon proximal movement of the deployment member 30. By virtue of the material from which the web 50 is formed causes the web 50 to at least partly engage with surrounding tissue of a patient once deployed and resist proximal motion so as to assist in the disengagement of the deployment member 30.

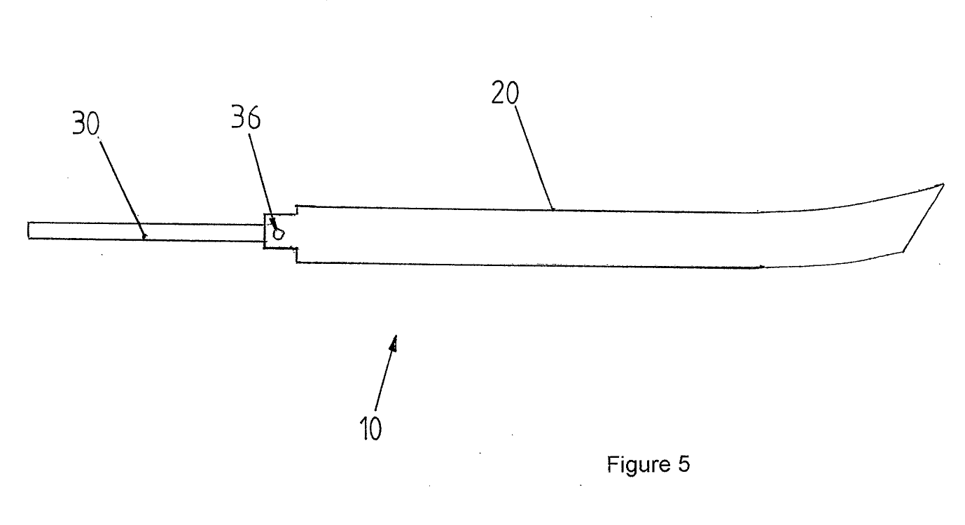

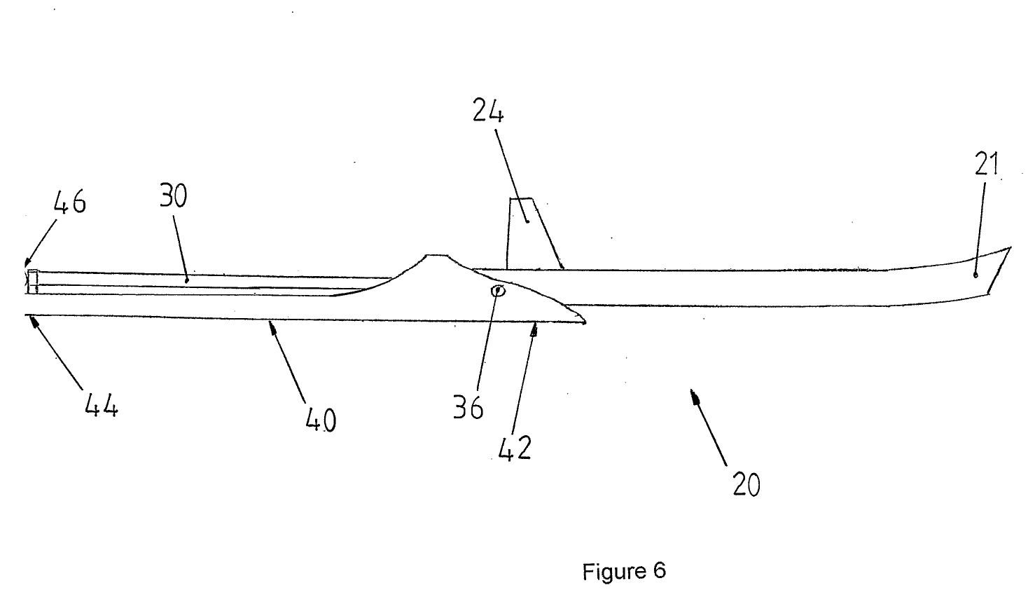

FIG. 5 depicts a further embodiment of the device 10 whereby a locking means 36 is provided such that the axial motion of the deployment member 30 relative to the carrier member 20 is fixed. The locking means 36 may be any means, for example a pin passing through both the carrier member 20 and the deployment member 30. Alternatively, the locking means may be a complimentary fit between the carrier member 20 and the deployment member 30 at a particular predetermined axial displacement. It will be appreciated that any manner in which the relative movement between the carrier member 20 and the deployment member 30 is prevented would constitute a locking means.

Referring to FIG. 6, a still further embodiment of the present invention is shown whereby the device 10 further comprises a generally elongate breach member 40 which extends proximally from the carrier member 20 and is slidingly engaged with the carrier member 20. A locking means 46 is provided to affix the deployment member 30 relative to the breach member 40. When the locking means 46 is engaged, the carrier member 20 can be withdrawn proximally whilst the breach member 40 and the deployment member 30 stay stationary with respect to each other, and a web may be deployed from the distal end 21 of the carrier member 20. As will be appreciated, the carrier member 20 may be placed at a location at which deployment of a web is desired. Once the carrier member 20 and consequently the web are at the desired locations, proximal withdrawal of the carrier member 20 deploys the web at the desired location. In this embodiment as with that as discussed with reference to FIG. 5, a locking means 36 is provided so as to lock the carrier member 20 relative to the deployment member 30. This allows for ease of delivery to the desired surgical site. The locking means 36 then must be disengaged prior to deployment of the web.

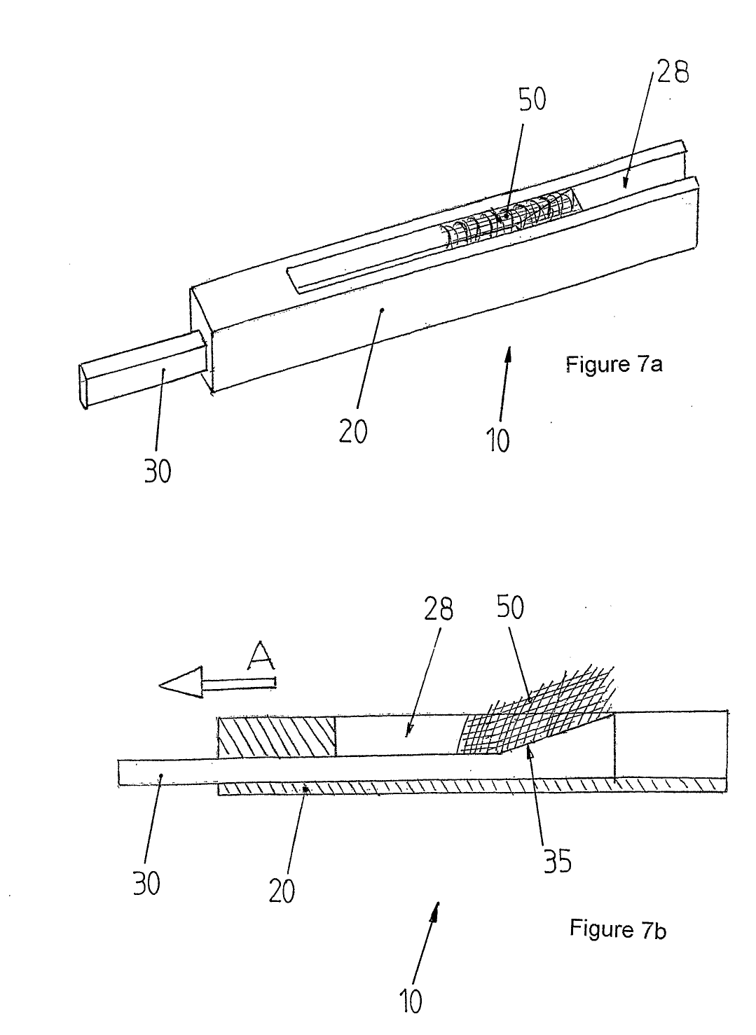

Referring to FIG. 7a and FIG. 7 b, yet another embodiment of the present invention is shown. FIG. 7a shows a perspective view of a device 10 wherein the passage 28 is open at one side of the carrier member 20 and extends a portion of the length of the carrier member 20. A web 50 is housed within the passage 28 such that when the deployment member 30 is advanced proximally as shown in sectional view FIG. 7b and denoted by direction arrow A, a wedge-shaped portion 35 located at the distal end 32 of the deployment member 30 drives the web 50 from the passage 28 such that the web 50 is deployed. It should be appreciated that numerous variations and embodiments exist whereby a web 50 is deployed from a passage 28 by relative movement between a deployment member 30 and a carrier member 20 without departing from the spirit and scope of the present invention.

The passage in the carrier member may be open along the entire length of the carrier member, so that the passage defines a visual path along which the surgeon performing the operation may look to assess whether the web has been properly deployed.

The present invention may be used in the treatment of urinary incontinence of women. Unlike the devices of the prior art, the method of the present invention does not make use a sub-urethral sling, and the device of the present invention does not allow deployment of a sub-urethral sling as used in the prior art. Rather, the present invention is used for the deployment of an elongate surgical web adjacent to the pubo-urethral ligament bilaterally which augments, bolsters or strengthens ligaments which are at least partially depleted or damaged, which assists in the treatment of urinary incontinence of women.

In contrast to techniques and devices for performing a sub-urethral sling procedure for the treatment of urinary incontinence, the present invention does not require the use of two supra-pubical incisions and as such, is less invasive than the procedures of the prior art. Consequently, the present invention does not result in the presence of unsightly scarring and being less invasive, has a lower infection risk. Furthermore, the present surgical approach required by the present invention reduces the likelihood of perforation or puncturing of the bladder of a patient, an undesirable complication which requires intra-operative surgical repair.

A less invasive procedure requiring less operating time as provided by the present invention and a device for performing such a procedure has significant advantages and benefits over the procedures and devices for performing such operations of the prior art. Less disruption and internal trauma to the patient suggests expedited healing and recovery. Furthermore, the reduced procedural steps required by to be performed a surgeon performing the above procedure with the device of the present invention, and the significant reduction of risk of perforation and damage to internal organs provides the surgeon with increased confidence and a lower likelihood of intra-operative complication. Furthermore, a more time effective procedure for the relatively commonly occurring complaint of urinary incontinence is thought to reduce costs on the community at large by less time in operating theatre and less post-operative management. The benefit of minimally invasive surgery is, of course, a significant benefit to a patient in recovery time, and time to return to normal daily routine.

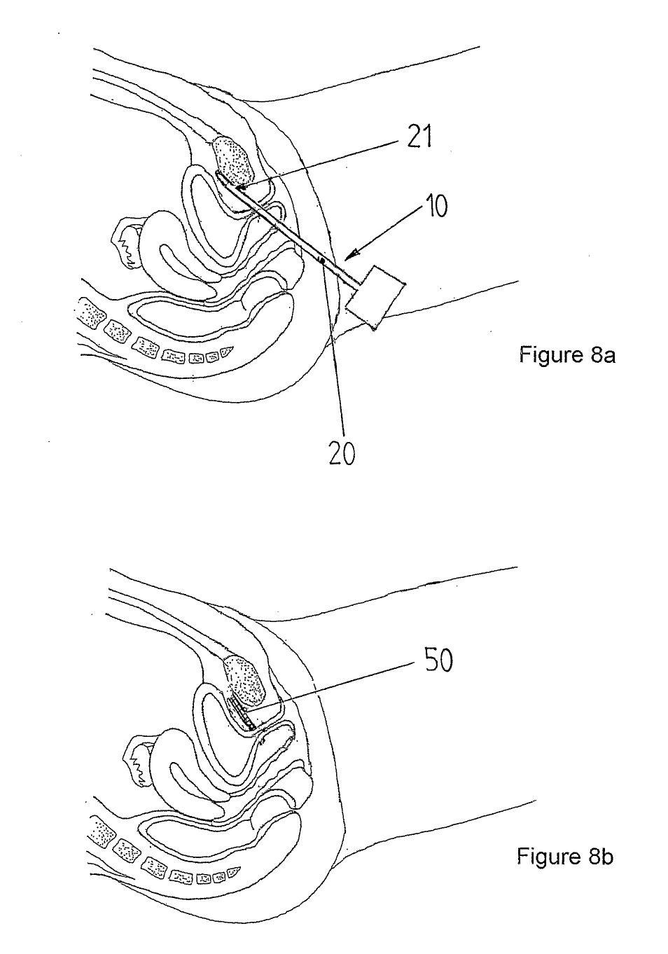

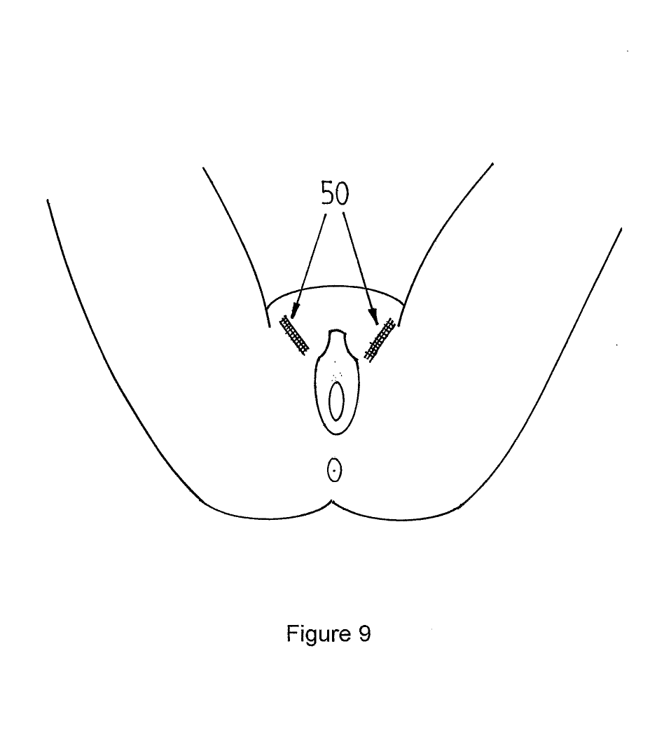

FIG. 8 shows a sagittal view through a female patient showing the location to which a surgical web is delivered and deployed, for the treatment of urinary incontinence in accordance with the present invention. As shown in FIG. 8a, a device 10 according to the present invention carrying a surgical web 50 within its lumen is inserted into the patient such that the distal end 21 of the carrier member 20 is located adjacent the pubo-urethral ligament of the patient such that the surgical web 50 is delivered to the surgical site.

The cutting tip 22 of the carrier member 20 provides assistance in separating the tissue of the patent, thus allowing the surgeon to more readily locate the distal end 21 of the carrier member adjacent the required surgical site within the patient.

When the deployment member 30 is moved relatively to the carrier member by maintaining the carrier member 20 stationary and deploying the web 50 distally, the cutting tip 38 of the deployment member 30 allows for separating of the tissue and further advancement of the web 50 via the deployment member 30 to the surgical site.

Upon deployment of the web 50, similarly as discussed above with reference to the preceding embodiments and preceding figures, either by maintaining the deployment member 30 or the carrier member 20 stationary with respect to the other, the web 50 is deployed at an appropriate surgical site as shown in FIG. 8b so as to strengthen or augment existing deficient ligaments. The cutting tip 38 of the deployment member 30 allows the deployment member 30 to be readily advanced.

Such a procedure may be performed bilaterally should ligaments on both sides require strengthening. As will be appreciated, varying surgical approaches may be used depending upon the particular ligaments that need strengthening. The procedure as described with reference to FIG. 8 is performed through a single incision, the single incision allowing delivery and deployment of the web 50 to both sides or aspects of the patient.

FIG. 9 depicts the locations at which the web 50 is located with respect to the patient's anatomy. As shown, in this embodiment of the present invention, the surgical web 50 is delivered and deployed such that it extends from an inferior medial point to a more superior and lateral point. It will appreciated that variations upon the location and the degree of inclination of the web 50 is taken to fall within the spirit and scope of the invention as claimed, and that the location is the web 50 is dependent upon the location which is required for augmentation or bolstering of deficient muscle or ligament tissue.

It is also to be understood that the device of the present invention, of course may be used to deliver and deploy a surgical web to other parts of a patient's body depending upon the medical condition of the patient. Modifications to the shape of the carrier member 20 may be required such as bends and angles so that a web 50 may be delivered to particular surgical sites without departing from the scope of the present invention.

It will be understood that the invention disclosed and defined herein extends to all alternative combinations of two or more of the individual features mentioned or evident from the text or drawings. All of these different combinations constitute various alternative aspects of the invention.

The foregoing describes embodiments of the present invention and modifications, obvious to those skilled in the art can be made thereto, without departing from the scope of the present invention.

1. A device for delivery and deployment of an elongate web into a patient, the device comprising:
a generally elongate carrier member adapted to be inserted into a body cavity or tissue of a patient, the carrier member having a proximal end and a distal end, and a passage extending along the carrier member at least part way along its length; and
an elongate deployment member having a distal end and a proximal end and being shaped and configured to be located in the passage and to be moved axially along the passage from a position adjacent the proximal end of the carrier member;
wherein the relative shape and configuration of the passage and the deployment member are such that a web is able to be housed in said passage when said deployment member is located in the passage; and
axially moving the carrier member and deployment member relative to each other in use being adapted to cause the web to be deployed from the passage into a patient.

2. A device according to claim 1, wherein the carrier member is of generally tubular configuration.

3. A device according to claim 1, wherein an engagement means is formed on or adjacent the distal end of said deployment member for engaging with the web and enabling the deployment member to carry the web out of the passage as the carrier member and deployment member are moved relative to each other.

4. A device according to claim 3, wherein the engagement means of said deployment member is adapted to disengage from the web when the deployment member is retracted from the patient in use.

5. A device according to claim 1, wherein said carrier member is circular in cross-sectional shape.

6. A device according to claim 1, wherein said carrier member is ovoid or elliptical in cross-sectional shape.

7. A device according to claim 1, wherein the carrier member is curved at least part way along its length.

8. A device according to claim 7, wherein the deployment member is laterally flexible.

9. A device according to claim 1, wherein said carrier member and said deployment member are adapted to be locked together to prevent relative movement during insertion of the carrier member into a patient.

10. A device according to claim 1, further comprising a generally elongate breach member, said breach member adapted to slidingly engage with said carrier member and extend away from the proximal end of the carrier member.

11. A device according to claim 10, wherein said deployment member and said breach member are adapted to be locked together.

12. A device according to claim 1, wherein the passage is an open channel and said deployment member has a portion inclined to the longitudinal axis of the carrier member such that relative motion between said carrier member and said deployment member drives the web member from the passage of the carrier member.

13. A device according to claim 1, wherein the elongate carrier member has a cutting tip formed at the distal end thereof.

14. A device according to claim 1, further comprising an elongate cutting member axially slidable in the passage of said carrier member so as to extend beyond the distal end of said carrier member, said cutting member having a distal end having cutting tip formed thereon, and a proximal end, the cutting member being removable from said passage after the carrier member has been operatively inserted into a patient in use.

15. A device according to claim 14, wherein the cutting tip of said cutting member includes at least one pair of opposite faces extending away from the proximal end of the cutting member and meeting at an apex.

16. A device according to claim 1, wherein the device is adapted to deploy a web elongate in shape and formed from a biocompatible polymeric material.

17. A device according to claim 16, wherein the web is textured so as to allow ingrowth of tissue.

18. A method for treating incontinence in a patient, the method comprising the steps of:
positioning a first elongate web adjacent to the pubo-urethral ligament of a patient on a first side; and
positioning a second elongate web adjacent to the pubo-urethral ligament of a patient on the contra-lateral side to the first elongate web;
wherein the first and the second elongate webs are formed by a material having a texture so as to allow ingrowth of tissue; and
the first and the second elongate webs are positioned at paraurethral locations within the body of the patient.

19. A method according to claim 18, wherein the first web and the second web are introduced into the body of the patient through a single access incision.

20. A method for treating incontinence in a patient, the method comprising the steps of:
positioning a first elongate web adjacent to the pubo-urethral ligament of a patient on a first side; and
positioning a second elongate web adjacent to the pubo-urethral ligament of a patient on the contra-lateral side to the first elongate web;
wherein the first and the second elongate webs are formed by a material having a texture so as to allow ingrowth of tissue; and
the first and the second elongate webs are positioned at paraurethral locations within the body of the patient, wherein the first web and the second web are introduced into the body with a device according to claim 1.

21. A method of treating a patient by positioning an elongate web of material at a selected location within the patient, the method including the steps of:
inserting a generally tubular carrier member into the patient, the carrier member having a proximal end and a distal end and a deployment passage extending between said ends, the carrier member being inserted to a position in the patient such that the distal end is at or adjacent said selected location; and
deploying said elongate web to said selected location through said deployment passage, using an elongate deployment member passing down said passage.

22. A method according to claim 21, wherein said step of deploying said elongate web is carried out by positioning said web at the distal end of the carrier member, keeping the web at that position by holding the deployment member stationary, at least partially withdrawing the carrier member from the patient to thereby leave the deployment member and web at said selected location, and fully withdrawing the carrier member and deployment member from the patient, leaving the web in said selected location within the patient.

23. A method according to claim 21, wherein the selected location is the muscle of the vaginal wall of a patient.

24. A method according to claim 21, wherein the method is for treatment of a patient having vault or vaginal prolapse.

25. A method according to claim 21, wherein the selected location is the pubu-urethral ligament of a patient.

26. A method according to claim 21, wherein the method is for treatment of a patient having stress-induced incontinence.

27. A method of treating a patient by positioning an elongate web of material at a selected location within the patient the method including the steps of:
inserting a generally tubular carrier member into the patient, the carrier member having a proximal end and a distal end and a deployment passage extending between said ends, the carrier member being inserted to a position in the patient such that the distal end is at or adjacent said selected location; and
deploying said elongate web to said selected location through said deployment passage, using an elongate deployment member passing down said passage, wherein the elongate web is delivered to and deployed at said selected location using a device according to claim 1.

28. A method according to claim 21, wherein the elongate web is adapted to allow tissue ingrowth.

29. A method according to claim 21, wherein the elongate web is provided with mechanical properties so as to augment the tissue of the patient at the selected location.

30. A method according to claim 21, wherein the elongate web includes at least one pharmaceutical substance for delivery of said pharmaceutical to said selected location.

31. (canceled)

32. (canceled)

33. (canceled)
